(19) United States
(12) Patent Application Publication
(10) Pub. No.: US 2010/0240767 A1
(43) Pub. Date: Sep. 23, 2010

(54) Melanogenesis inhibitiuon by 3,5-dimethoxy-4'-hydroxystilbenes and cosmeceutical compositions thereof

(76) Inventors: MUHAMMED MAJEED, PISCATAWAY, NJ (US); BEENA BHAT, BANGALORE (IN); GEETHA KANHANGAD-GANGADHARAN, BANGALORE (IN); SUSMITHA ANAND, BANGALORE (IN)

Correspondence Address: SABINSA CORPORATION, 20 Lake Drive, East Windsor, NJ, 08520, US

(21) Appl. No.: 12/408,808

(22) Filed: Mar. 23, 2009

(51) Int. Cl. A61K 31/085; A61P 17/00

(52) U.S. Cl. 514/720

(57) ABSTRACT

Disclosed is the cosmeceutical potential of 3,5-dimethoxy-4′-hydroxystilbene in terms of its melanogenesis inhibitory and photo protective activities. Also disclosed does a topical melanogenesis inhibitory composition comprising 0.01 to 50% by weight of 3,5-dimethoxy-4′-hydroxystilbene.

### BACKGROUND OF THE INVENTION

1. Field of the Invention

The invention in general relates to 3,5-dimethoxy-4′-hydroxystilbene. More specifically, the invention discloses the cosmeceutical potential of 3,5-dimethoxy-4′-hydroxystilbene (pterostilbene) in terms of its melanogenesis inhibitory and photo protective activities and compositions thereof.

2. Description of Prior Art

Pterostilbene is a stilbenoid belonging to a group of compounds called phytoalexins which are produced by plants in response to stress It is thought to be the key compound found predominantly in blueberries and grapes known to exhibit anti-cancer, anti-hypercholesterolemia, anti-hypertriglyceridemia, anti-diabetic and anti-fungal potential.

It is the primary objective of the present invention to disclose novel melanogenesis inhibitory activity of pterostilbene.

It is another objective of the present invention to disclose a melanogenesis inhibitory composition comprising 3,5-dimethoxy-4′-hydroxystilbene, wherein said composition by weight from about 0.01% to about 50% 3,5-dimethoxy-4′-hydroxystilbene.

It is yet another objective of the present invention to disclose the use of 3,5-dimethoxy-4′-hydroxystilbene in the manufacture of a medicament to treat skin-hyper pigmentation.

Further, the present invention also discloses a method of treating skin hyper-pigmentation.

The present invention fulfills the aforementioned objectives and provides further related advantages.

### SUMMARY OF THE INVENTION

The invention relates to the cosmeceutical potential of 3,5-dimethoxy-4′-hydroxystilbene in terms of its melanogenesis inhibitory and photo protective activities. Also disclosed does a topical melanogenesis-inhibitory composition comprising 0.01 to 50% by weight of 3,5-dimethoxy-4′-hydroxystilbene.

Other features and advantages of the present invention will become apparent from the following more detailed description which illustrates by way of example, the principle of the invention.

### DESCRIPTION OF THE PREFERRED EMBODIMENT

In another most preferred embodiment the present invention relates to a melanogenesis inhibitory composition comprising 3,5-dimethoxy-4′-hydroxystilbene represented by STR#1, wherein said composition by weight from about 0.01% to about 50% 3,5-dimethoxy-4′-hydroxystilbene.

In yet another most preferred embodiment, the present invention relates to the use of 3,5-dimethoxy-4′-hydroxystilbene represented by STR#1, in the manufacture of a medicament to treat skin-hyper pigmentation.

In yet another most preferred embodiment, the present invention relates to a method of treating skin hyper-pigmentation, said method comprising the topical application on affected areas of the skin in need of such treatment, a composition comprising by weight from about 0.01% to about 50% 3,5-dimethoxy-4′-hydroxystilbene represented by STR#1.

In yet another most preferred embodiment, the present invention relates to a method of treating skin hyper-pigmentation, said method comprising the topical application on affected areas of the skin in need of such treatment, a composition comprising by weight from about 0.01% to about 50% 3,5-dimethoxy-4′-hydroxystilbene represented by STR#1, the said stilbene being obtained from Pterocarpus marsupium extract in commercially significant purity of at least 90%. The stilbene 3,5-dimethoxy-4′-hydroxystilbene was obtained by solvent extraction of Pterocarpus marsupium. The solvents used were ethyl acetate and hexane. Other solvents of similar polarity can be used. Supercritical carbon di-oxide, scCO2, can also be used for removal of non-polar constituents from Pterocarpus marsupium. Alternatively, pterostilbene can be obtained by synthesis as exemplified in U.S. Pat. No. 7,253,324

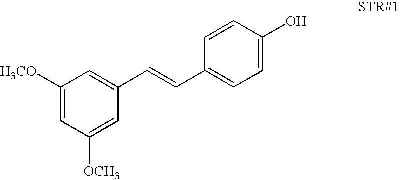

### EXAMPLE I

The mushroom tyrosinase inhibitory activity of 3,5-dimethoxy-4′-hydroxystilbene was studied in a micro plate by incubating varying concentrations of the sample with 400 U/ml of mushroom tyrosinase and then by the addition of 3.53 mM of L-tyrosine substrate. The absorbance at the 10th minute of the reaction was read in a Fluostar Optima micro plate reader at 492 nm. 3,5-dimethoxy-4′-hydroxystilbene showed a significant inhibition of mushroom tyrosinase with an IC50 value of 7 μg/ml.

### EXAMPLE II

3,5-dimethoxy-4′-hydroxystilbene was studied for melanogenesis inhibition in B16F1 mouse melanoma cell line. B16F1 cells were induced with 0.5 nM MSH (Melanocyte stimulating hormone) and then treated with varying concentrations of the sample. Sample treatment was given thrice after every 72 hrs. On the 9th day of treatment, the melanin from the cells was extracted with 1N NaOH and the absorbance was read at 405 nm in a Fluostar Optima micro plate reader. 3,5-dimethoxy-4′-hydroxystilbene showed a significant inhibition of melanogenesis with an IC50 value of 0.5 μg/ml. 3,5-dimethoxy-4′-hydroxystilbene surprisingly showed about 6 times higher potential than Glabridin.

### EXAMPLE III

3,5-dimethoxy-4′-hydroxystilbene was studied for UV protection in Swiss 3T3 mouse fibroblast cells. Swiss 3T3 fibroblast cell monolayers were exposed to UV A (0.5 J/cm2) and UV B (0.05 J/cm2) in the presence and absence of sample treatment at varying concentrations. After UV exposure, the cells were incubated in CO2 incubator for 72 hours and the cell viability was determined by SRB (Sulphorhodomine B) dye staining. The UV protection is determined as the enhanced percentage of viable sample treated cells as compared to the untreated cells after UV exposure. 3,5-dimethoxy-4′-hydroxystilbene showed a significant UV protection of 32% as compared to the untreated cells.

### EXAMPLE IV

Pterostilbene Compositions of the Present Invention (Illustrative Examples)

### Prototype Formula for Depigmentation Cream

| | Ingredients | % w/w | Function |
|---|---|---|---|
| A | Glyceryl mono stearate | qs | Emulsifying agent |
| | Isopropyl palmitate | qs | Emollinet |
| | PEG 100 stearate | qs | Emolllient |
| | Stearic acid | qs | Emulsifying agent |
| | Capric Caprylic Triglyceride | qs | Emollient |
| | Propyl Paraben | | Preservative |
| B | Propylene glycol | qs | Humectant |
| | Tetrasdium EDTA | qs | Chelating agent |
| | Imidurea | qs | Preservative |
| | Methyl Paraben | qs | Preservative |
| | Demineralised water | up to 100 | Solvent |
| C | Pterostillbene | 0.1 | Active |
| | Arbutin | 1.0 | Active |
| | Licorice 40% CA | 0.1 | Active |
| | Ethanol | qa | Solubilizer |
| D | 20% Sodium Hydroxide Solution | qs | Neutralizer |
| | (if required to adjust the pH) | | |
| | DC 3021 | qs | Silicon |
| | Salcare SC 91 | qs | Viscosity modifier |
| E | Fragrance | qs | Fragrance |

### Properties:

Appearance: Light brown colored emulsion
pH: 5.5-7.5

Alternative formulation
Ceteareth-25-2.0%
Glyceryl Stearate-4.0%
Stearyl Alcohol-2.0%
Ethyl hexyl Stearate-8.5%
Caprylic/Capric triglyceride-5.5%
Water-75.0%
Kathon CG-2-3 Tr
Pterostilbene (90%)-1%

While the invention has been described with reference to a preferred embodiment, it is to be clearly understood by those skilled in the art that the invention is not limited thereto. Rather, the scope of the invention is to be interpreted only in conjunction with the appended claims.

We claim:

1. A melanogenesis inhibitory composition comprising 3,5-dimethoxy-4′-hydroxystilbene represented by STR#1, wherein said composition by weight from about 0.01% to 50% 3,5-dimethoxy-4′-hydroxystilbene.

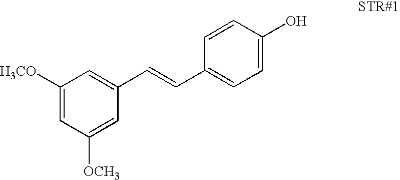

2. The use of 3,5-dimethoxy-4′-hydroxystilbene represented by STR#1, in the manufacture of a medicament to treat skin-hyper pigmentation.

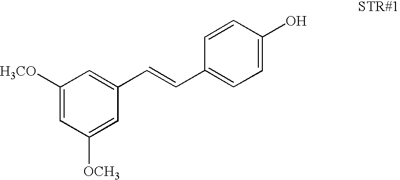

3. A method of treating skin hyper-pigmentation, said method comprising the topical application on affected areas of the skin in need of such treatment, a composition comprising by weight from about 0.01% to 50% 3,5-dimethoxy-4′-hydroxystilbene represented by STR#1.

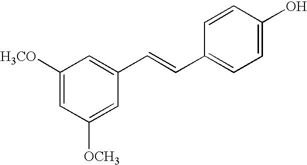

4. A melanogenesis inhibitory composition comprising 3,5-dimethoxy-4′-hydroxystilbene as claimed in claim 1 wherein 3,5-dimethoxypterostilbene is obtained from Pterocarpus marsupium in purity levels above 50% w/w.

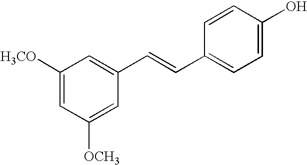

5. The use of 3,5-dimethoxy-4′-hydroxystilbene as claimed in claim 2 wherein 3,5-dimethoxy-4′-hydroxystilbene is obtained from Pterocarpus marsupium in purity levels above 50% w/w.

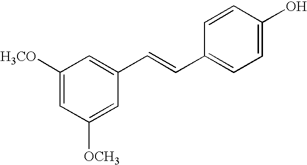

6. The use of 3,5-dimethoxy-4′-hydroxystilbene as claimed in claim 3 wherein 3,5-dimethoxy-4′-hydroxystilbene is obtained from Pterocarpus marsupium in purity levels above 50% w/w. [chemical structure]
